(19) United States
(12) Patent Application Publication
(10) Pub. No.: US 2021/0113173 A1
(43) Pub. Date: Apr. 22, 2021

(54) COMPUTED TOMOGRAPHY (CT) SYSTEM AND CT IMAGE RECONSTRUCTION METHOD

(71) Applicant: HEFEI UNIVERSITY OF TECHNOLOGY, Hefei (CN)

(72) Inventors: Xiaojia WANG, Hefei (CN); Kuo DU, Hefei (CN); Keyu ZHU, Hefei (CN); Sheng XU, Hefei (CN)

(21) Appl. No.: 16/897,515

(22) Filed: Jun. 10, 2020

(30) Foreign Application Priority Data
Oct. 22, 2019 (CN) 201911006017.7

(51) Int. Cl. A61B 6/00; G16H 30/40; G16H 50/20; G16H 15/00; A61B 6/03; A61M 5/00; A61K 49/04; G06T 7/00; G06T 11/00

(52) U.S. Cl. CPC A61B 6/5235; G06T 2207/30016; G16H 50/20; G16H 15/00; A61B 6/035; A61B 6/462; A61B 6/4411; A61M 5/007; A61B 6/501; A61B 6/504; A61B 6/5258; A61K 49/0438; G06T 7/0012; G06T 11/003; G06T 2207/10081; G06T 2207/30096; G16H 30/40

(57) ABSTRACT

The present invention discloses a computed tomography (CT) system and a CT image reconstruction method. The method is implemented by an information processing system module, a display terminal module, an image reconstruction system module and a CT system module. The information processing system module is composed of an information receiving system module, an information sending system module and an information processing system module. During scanning, different nodes are arranged and quickly scanned to obtain data of a node, and then data of different nodes is synchronized, collected and summarized, so that a reconstruction unit can be analyzed, an objective function can be optimized, and data can be quickly analyzed and processed. In this way, the CT system and the CT image reconstruction method resolve the problems that an existing CT system and CT image reconstruction method have high requirements for projection data sets, and scanning requires a long time.

### TECHNICAL FIELD

The present invention relates to a technical field related to computed tomography (CT) systems, and particularly relates to a CT system and a CT image reconstruction method.

### BACKGROUND

With the development of medical imaging, the medical imaging technology has been widely applied to clinical judgment and treatment. Common medical imaging equipment includes CT systems and ultrasound systems. CT is a procedure that uses X-rays to scan a specific area of a human body according to a certain thickness. Because human tissues vary in the capability of absorbing X-rays, a computer can be used to reconstruct a cross-sectional image. With regard to psychology, a CT system can be used for checking perception, thinking, emotion, willpower behaviors, sleep, psychological development, lies, interpersonal relationship, learning ability, personal style, wedding and family problems, psychological defense mechanisms, body and mental diseases and the like, thereby comprehensively measuring psychological health and quality.

An existing CT system and CT image reconstruction method have the following problems: 1) optimal reconstruction available only when a large amount of projection data is acquired at a uniform and dense angle; 2) obvious streak artifacts existing when there are insufficient projection angles; 3) high requirements for a projection data set, specifically, the data set must be accurate and continuous; 4) long CT scanning time; 5) large dose and motion artifacts.

### SUMMARY

An objective of the present invention is to provide a CT system and a CT image reconstruction method to solve the above problems that the existing CT system and CT image reconstruction method have extremely high requirements for the projection data set, and CT scanning requires a long time.

To achieve the above objectives, the present invention provides the following technical solution.

Disclosed are a CT system and a CT image reconstruction method. The method is implemented by an information processing system module, a display terminal module, an image reconstruction system module and a CT system module. The information processing system module is composed of an information receiving system module, an information sending system module and an information processing system module. The information receiving system module is used for receiving and storing information after inspection; the information sending system module is used for sending the information after inspection; and the information processing system module is used for processing needed information and unnecessary information differently.

Preferably, the display terminal module is composed of a main control computer, a terminal machine, a photoelectric reader, a report printer and a display, where the main control computer has a computer operation function; the terminal machine has a computer control function; the photoelectric reader has a viewing function; the report printer can be used for printing reports and a display screen in the display terminal module can be used for playing to-be-viewed information.

Preferably, the image reconstruction system module is composed of a data statistics module, a data analysis module and a data modeling module, where the data statistics module is used for performing statistics on data after scanning; the data analysis module is used for analyzing the data after scanning; and the data modeling module is used for performing modeling on data related to constructed images.

Preferably, the CT system module is composed of a detection system module, an analysis system module and a converter processing module, where the detection system module is used for detecting a patient; the analysis system module is used for analyzing data after detection; and the converter processing module is used for converting data after detection.

Preferably, operation steps of the image reconstruction system module are as follows: step 1: establishing a CT system analysis model;

step 2: inputting and summarizing detected data, and analyzing and processing the data through the CT system module;

step 3: acquiring imaging system parameters of CT equipment and projection data complying with a low-dose CT scanning protocol, constructing a priori data statistical model based on structural features of projection data and chord graph data and requirements in actual applications;

step 4: constructing a statistical model for generating projection data according to data induction and collection through a statistical law of the imaging process;

step 5: establishing different nodes according to the statistical model, and planning a reconstruction unit according to the nodes;

(1) scanning a specific area of a human body according to a certain thickness with X-ray beams, and performing calculation on information obtained after scanning to obtain an X-ray attenuation coefficient or absorbing coefficient, which is further arranged to form a digital matrix, of each voxel, where the digital matrix can be stored in a magnetic disc or an optical disc; and converting each numeral in the digital matrix into small black and white square blocks, namely pixels, with different gray scales through a digital/analog converter, and arranging the pixels in a matrix to form a CT image;

(2) receiving X-rays transmitted through the area by a detector, converting the X-rays into visible light, converting the visible light into electrical signals through photoelectric conversion, converting the electrical signals to numerals by an analog/digital converter, inputting the numerals into a computer to be processed, and forming an image by the processed data;

step 6: synchronizing, collecting and summarizing data of different nodes, analyzing a reconstruction unit, and optimizing an objective function, where the objective function can be optimized according to general objective function optimization methods, such as a gradient descent method;

step 7: adjusting the proportional relationship between the sectional size of a to-be-detected object and real-time images of the CT system to enable images formed by the CT system and related to the same section to correspond to each other and to enable the to-be-detected object to move in a certain linear direction, and then scanning and imaging the to-be-detected object by the CT system to obtain second combined coordinates of the real-time images of the to-be-detected object; and

step 8: fusing the reconstructed image of the to-be-detected object and the real-time images of the to-be-detected object to obtain a fused image, and then performing CT image reconstruction according to the data to obtain an output result.

Preferably, the detection system module has the following three working modes:

(1) unenhanced scanning, which refers to non-contrast-enhanced scanning or non-contrast scanning, and is generally performed first;

(2) enhanced scanning, which uses a high-pressure injector to perform transvenous injection of a water-soluble organic iodine agent, such as 60 ml of 60%-76% meglumine diatrizoate before scanning; after the concentration of iodine in blood increases, the concentration of iodine in a normal organ and the concentration of iodine in a lesion can be different, and a density difference is formed, which makes the development of the lesion clearer; and enhanced scanning mainly includes bolus injection and intravenous therapy; and

(3) angiography scanning, which conducts angiography on an organ or structure before scanning: for example, a cerebral cistern and a small tumor therein can be clearly displayed by injecting 8-10 ml of iotrolan or 4-6 ml of air into the cerebral cistern for cisternography rescanning, namely, cisternography CT scanning.

Compared with the prior art, the CT system and the CT image reconstruction method have the following beneficial effects:

during scanning, different nodes are arranged and quickly scanned to obtain data of a node, and then data of different nodes is synchronized, collected and summarized, so that a reconstruction unit can be analyzed, an objective function can be optimized, and data can be quickly analyzed and processed. In this way, the present invention effectively resolves the problems that an existing CT system and CT image reconstruction method have high requirements for projection data sets, and scanning requires a long time.

### DETAILED DESCRIPTION

The present invention provides the following technical solution.

Disclosed are a CT system and a CT image reconstruction method. The method is implemented by an information processing system module, a display terminal module, an image reconstruction system module and a CT system module. The information processing system module is composed of an information receiving system module, an information sending system module and an information processing system module. The information receiving system module is used for receiving and storing information after inspection; the information sending system module is used for sending the information after inspection; and the information processing system module is used for processing needed information and unnecessary information differently.

The display terminal module is composed of a main control machine, a terminal machine, a photoelectric reader, a report printer and a display device, where the main control machine has a computer operation function; the terminal machine has a computer control function; the photoelectric reader has a viewing function; the report printer has a printing function; and the display screen has the function of playing what is to be viewed.

The image reconstruction system module is composed of a data statistics module, a data analysis module and a data modeling module, where the data statistics module is used for performing statistics on the data after scanning; the data analysis module is used for analyzing the data after scanning; and the data modeling module is used for performing modeling on data related to constructed images.

The CT system module is composed of a detection system module, an analysis system module and a converter processing module, where the detection system module is used for detecting the patient, the analysis system module is used for analyzing the data after detection, and the converter processing module is used for converting the data after detection.

The operation steps of the image reconstruction system module are as follows:

step 1: establishing a CT system analysis model;

step 2: inputting and summarizing detected data, and then analyzing and processing the detected data through the CT system module;

step 3: acquiring imaging system parameters of CT equipment and projection data complying with a low-dose CT scanning protocol, and constructing a priori data statistical model based on structural features of projection data and chord graph data and requirements in actual applications;

step 4: constructing a statistical model for generating projection data according to data induction and collection through a statistical law of the imaging process;

step 5: establishing different nodes according to the statistical model, and planning a reconstruction unit according to the nodes:

(1) scanning a specific area of a human body according to a certain thickness with X-ray beams, performing calculation on information obtained after scanning to obtain an X-ray attenuation coefficient or absorbing coefficient, which is further arranged to form a digital matrix, of each voxel, where the digital matrix can be stored in a magnetic disc or an optical disc; and converting each numeral in the digital matrix into small black and white square blocks, namely pixels, with different gray scales through a digital/analog converter, and arranging the pixels in a matrix to form a CT image; and

(2) receiving X-rays transmitted through the area by a detector, converting the X-rays into visible light, converting the visible light into electrical signals through photoelectric conversion, converting the electrical signals to numerals by an analog/digital converter, inputting the numerals into a computer to be processed, and forming an image by the processed data;

step 6: synchronizing, collecting and summarizing data of different nodes, analyzing a reconstruction unit, and optimizing an objective function, where the objective function can be optimized according to general objective function optimization methods, such as a gradient descent method;

step 7: adjusting the proportional relationship between the sectional size of a to-be-detected object and real-time images of the CT system to enable images formed by the CT system and related to the same section to correspond to each other and to enable the to-be-detected object to move in a certain linear direction, and then scanning and imaging the to-be-detected object by the CT system to obtain second combined coordinates of the real-time images of the to-be-detected object; and

step 8: fusing the reconstructed image of the to-be-detected object and the real-time images of the to-be-detected object to obtain a fused image, and then performing CT image reconstruction according to the data to obtain an output result.

The detection system module has the following three working modes:

(1) unenhanced scanning, which refers to non-contrast-enhanced scanning or non-contrast scanning, and is generally performed first;

(2) enhanced scanning, which uses a high-pressure injector to perform transvenous injection of a water-soluble organic iodine agent, such as 60 ml of 60%-76% meglumine diatrizoate before scanning; after the concentration of iodine in blood increases, the concentration of iodine in a normal organ and the concentration of iodine in a lesion can be different, and a density difference is formed, which makes the development of the lesion clearer; and enhanced scanning mainly includes bolus injection and intravenous therapy; and

(3) angiography scanning, which conducts angiography on an organ or structure before scanning: for example, a cerebral cistern and a small tumor therein can be clearly displayed by injecting 8-10 ml of iotrolan or 4-6 ml of air into the cerebral cistern for cisternography rescanning, namely, cisternography CT scanning.

The working principle and application process of the present invention are as follows:

Since different human tissues differ in X-ray absorbing and transmittance, an instrument with extremely high sensitivity is used for measuring a human body; then acquired measurement data is input to and processed by an electronic computer, and therefore a sectional or vertical image of a checked area of the human body is shot to find out any tiny pathological change. Firstly, a specific area of a human body is scanned according to a certain thickness by X-ray beams, information obtained after scanning is subjected to calculation to obtain an X-ray attenuation coefficient or absorbing coefficient, which is further arranged to form a digital matrix, of each voxel, where the digital matrix can be stored in a magnetic disc or an optical disc; and each numeral in the digital matrix is converted into small black and white square blocks, namely pixels, with different gray scales through a digital/analog converter, and the pixels are arranged in a matrix to form a CT image; X-rays transmitted through the area are received by a detector and are converted into visible light, the visible light is converted into electrical signals through photoelectric conversion, the electrical signals are converted to numerals by an analog/digital converter and are input into a computer to be processed, the proportional relationship between the sectional size of a to-be-detected object and real-time images of the CT system is adjusted to enable images formed by the CT system and related to the same section to correspond to each other and to enable the to-be-detected object to move in a certain linear direction, and then the to-be-detected object is scanned and imaged to obtain second combined coordinates of the real-time images of the to-be-detected object; and the reconstructed image of the to-be-detected object and the real-time images of the to-be-detected object are fused to obtain a fused image, and then CT image reconstruction is performed according to the data to obtain an output result, and an image is formed by the processed data.

Although the examples of the present invention have been illustrated and described, it should be understood that those of ordinary skill in the art may make various changes, modifications, replacements and variations to the above examples without departing from the principle and spirit of the present invention, and the scope of the present invention is limited by the appended claims and their legal equivalents.

What is claimed is:

1. A computed tomography (CT) system and a CT image reconstruction method, the CT image reconstruction method being implemented by an information processing system module, a display terminal module, an image reconstruction system module and a CT system module, wherein the information processing system module is composed of an information receiving system module, an information sending system module and an information processing system module; the information receiving system module is used for receiving and storing information after inspection; the information sending system module is used for sending the information after inspection; and the information processing system module is used for processing needed information and unnecessary information differently;
the display terminal module is composed of a main control computer, a terminal machine, a photoelectric reader, a report printer and a display, wherein the main control computer has a computer operation function; the terminal machine has a computer control function; the photoelectric reader has a viewing function; the report printer has a printing function; and a display screen in the display terminal module can be used for playing what is to be viewed later;
the image reconstruction system module is composed of a data statistics module, a data analysis module and a data modeling module, wherein the data statistics module is used for performing statistics on data after scanning; the data analysis module is used for analyzing the data after scanning; and the data modeling module is used for performing modeling on data related to constructed images;
and the CT system module is composed of a detection system module, an analysis system module and a converter processing module, wherein the detection system module is used for detecting a patient; the analysis system module is used for analyzing data after detection; and the converter processing module is used for converting data after detection.

2. The CT system and the CT image reconstruction method according to claim 1, wherein
the operation steps of the image reconstruction system module are as follows:
step 1: establishing a CT system analysis model;
step 2: inputting and summarizing detected data, and then analyzing and processing the detected data through the CT system module;
step 3: acquiring imaging system parameters of CT equipment and projection data complying with a low-dose CT scanning protocol, and constructing a priori data statistical model based on structural features of projection data and chord graph data and requirements in actual applications;
step 4: constructing a statistical model for generating projection data according to data induction and collection through a statistical law of the imaging process;
step 5: establishing different nodes according to the statistical model, and planning a reconstruction unit according to the nodes;
(1) scanning a specific area of a human body according to a certain thickness with X-ray beams, and performing calculation on information obtained after scanning to obtain an X-ray attenuation coefficient or absorbing coefficient, which is further arranged to form a digital matrix, of each voxel, where the digital matrix can be stored in a magnetic disc or an optical disc; and converting each numeral in the digital matrix into small black and white square blocks, namely pixels, with different gray scales through a digital/analog converter, and arranging the pixels in a matrix to form a CT image;
(2) receiving X-rays transmitted through the area by a detector, converting the X-rays into visible light, converting the visible light into electrical signals through photoelectric conversion, converting the electrical signals to numerals by an analog/digital converter, inputting the numerals into a computer to be processed, and forming an image by the processed data;
step 6: synchronizing, collecting and summarizing data of different nodes, analyzing a reconstruction unit, and optimizing an objective function, where the objective function can be optimized according to general objective function optimization methods, such as a gradient descent method;
step 7: adjusting the proportional relationship between the sectional size of a to-be-detected object and real-time images of the CT system to enable images formed by the CT system and related to the same section to correspond to each other and to enable the to-be-detected object to move in a certain linear direction, and then scanning and imaging the to-be-detected object by the CT system to obtain second combined coordinates of the real-time images of the to-be-detected object; and
step 8: fusing the reconstructed image of the to-be-detected object and the real-time images of the to-be-detected object to obtain a fused image, and then performing CT image reconstruction according to the data to obtain an output result.

3. The CT system and the CT image reconstruction method according to claim 1, wherein the detection system module has the following three working modes:
(1) unenhanced scanning, which refers to non-contrast-enhanced scanning or non-contrast scanning, and is generally performed first;
(2) enhanced scanning, which uses a high-pressure injector to perform transvenous injection of a water-soluble organic iodine agent, such as 60 ml of 60%-76% meglumine diatrizoate before scanning; after the concentration of iodine in blood increases, the concentration of iodine in a normal organ and the concentration of iodine in a lesion can be different, and a density difference is formed, which makes the development of the lesion clearer; and enhanced scanning mainly includes bolus injection and intravenous therapy; and
(3) angiography scanning, which conducts angiography on an organ or structure before scanning: for example, a cerebral cistern and a small tumor therein can be clearly displayed by injecting 8-10 ml of iotrolan or 4-6 ml of air into the cerebral cistern for cisternography rescanning, namely, cisternography CT scanning.
